(19) United States
(12) Patent Application Publication
(10) Pub. No.: US 2021/0251335 A1
(43) Pub. Date: Aug. 19, 2021

(54) AN ASSEMBLY FOR PROVIDING FOOTWEAR WITH A PLURALITY OF REMOVABLE AND INTERCHANGEABLE FOOTBEDS

(71) Applicant: FRANKIE4 IP 2 Pty Ltd, Indooroopilly (AU)

(72) Inventor: Caroline Rose Haire, Samford (AU)

(73) Assignee: FRANKIE4 IP 2 Pty Ltd, Indooroopilly (AU)

(21) Appl. No.: 17/256,810

(22) PCT Filed: Jul. 4, 2019
(86) PCT No.: PCT/AU2019/050705

§ 371 Date: Dec. 29, 2020

(30) Foreign Application Priority Data
Jul. 6, 2018 (AU) 2018902465
Jun. 19, 2019 (AU) 2019902139

(51) Int. Cl. A43B 13/18

(52) U.S. Cl. CPC A43B 13/188

(57) ABSTRACT

An assembly for providing footwear with a plurality of removable and interchangeable footbeds. The assembly providing an item of footwear, a cushioning full length footbed adapted for insertion within the footwear to cooperate with the footwear to thereby produce a soft feel, and a supportive full length footbed adapted for insertion within the footwear to cooperate with the footwear to thereby produce a supportive feel. In use, the cushioning full length footbed is inserted into the item of footwear to provide a soft feeling underfoot, or the supportive full length footbed is inserted into the item of footwear to provide a support to reduce or prevent excessive pronation.

### FIELD OF THE INVENTION

The present invention relates to a footwear insert system and assembly for providing footwear with a plurality of removable and interchangeable footbeds. In particular, the invention relates to an improved footbed assembly for shoes to reduce or prevent excessive pronation during wear or to provide a cushioning or softening effect during wear.

### BACKGROUND

Reference to background art herein is not to be construed as an admission that such art constitutes common general knowledge.

Footwear inserts, commonly known as shoe inserts, footbeds or insoles, are removable items placed within a shoe, which can be used to support a portion of a foot (such as the heel or arch) or the entire length of the foot.

During running and gait, a wearer's shoe contacts the ground such that the foot lands on a lateral side of the heel first before the centre of the load moves to the medial side of the foot. During this process, the foot naturally pronates. However, in many people, the wearer's foot and ankle overpronate (i.e. the ankle rolls too far downward and inward with each step and continues to roll when the foot should be starting to push off). Overpronation is particularly common in people with “flat feet” but can occur in anyone. This overpronation can lead to injuries, particularly overuse type injuries, due to increased and repetitive stress on the muscles, tendons and ligaments of the foot and lower leg including the shin and knee as the limb rotates too far inwardly.

Making shoes that correct or prevent overpronation which are also comfortable to wear for extended periods is difficult. One reason is that while different people may have the same foot length, the feet can still vary in width. Thus, a foot of the same length may still be classified as a “narrow” foot, a “standard width” foot or a “wide” foot. A person's foot can also slightly change shape (e.g. slightly swell) over a day.

Shoes which provide support for overpronation are known. However, these shoes are usually more expensive than regular, unsupportive shoes, and the support is integrated with the shoe in the form of the midsole, sole or outsole. A problem with these shoes is that each foot of a user may have specific support requirements. Further, over time, the midsole will wear out and the entire shoe must then be replaced.

Additionally, in order to accommodate feet of varying widths, a large range of shoes having the same length but different widths are required. This is expensive and takes up considerable shelf space.

### OBJECT OF THE INVENTION

It is an aim of this invention to provide an assembly for providing footwear with a plurality of removable and interchangeable footbeds which overcomes or ameliorates one or more of the disadvantages or problems described above, or which at least provides a useful commercial alternative.

Other preferred objects of the present invention will become apparent from the following description.

### SUMMARY OF THE INVENTION

In one form, although it need not be the only or indeed the broadest form, there is provided an assembly for providing footwear with a plurality of removable and interchangeable footbeds, the assembly comprising:
an item of footwear;
a cushioning full length footbed adapted for insertion within the footwear to cooperate with the footwear to thereby produce a soft feel; and
a supportive full length footbed adapted for insertion within the footwear to cooperate with the footwear to thereby produce a supportive feel,
wherein in use the cushioning full length footbed is inserted into the item of footwear to provide a soft feeling underfoot; or
the supportive full length footbed is inserted into the item of footwear to provide a supportive to reduce or prevent excessive pronation.

Preferably, in use, the cushioning full length footbed and the supportive full length footbed are interchangeably insertable into the item of footwear.

Preferably, the supportive footbed comprises a medial side and a lateral side. Preferably, the supportive footbed comprises a support located along the medial side. Preferably, the support is a cushioned or contoured portion adapted to support an arch of a foot in the item of footwear to reduce or prevent excessive pronation.

Preferably, the assembly further comprises:
a forefoot insert arranged for insertion beneath the cushioning full length footbed or the supportive full length footbed within the footwear so that the cushioning full length footbed or the supportive full length footbed and the forefoot insert cooperate with the footwear to produce a narrow fitting,
wherein in use the cushioning full length footbed or the supportive full length footbed with the forefoot insert beneath a forward portion thereof is inserted into the item of footwear to provide a narrow fit in combination with the soft feel of the cushioning full length footbed or the supportive feel of the supportive full length footbed.

Preferably, the forefoot insert is attached to the cushioning full length footbed or the supportive full length footbed. More preferably, the forefoot insert is adhesively attached to the cushioning full length footbed or the supportive full length footbed.

Preferably, the item of footwear is an item of neutral footwear or neutral shoe. Neutral footwear may be a substantially flat or unsupportive footwear or shoe.

Preferably, the shoe is a neutral shoe, a shoe adapted for activity (e.g. running) or a casual shoe (e.g. sneakers). Alternatively, the shoe comprises a built-up or supportive sole, which may comprise a built-up or supportive midsole or outsole.

In another form, the invention resides in an assembly comprising:
a container;
a pair of shoes located in the container; and
at least two pairs of footbeds to provide a user with at least two fitting choices for the same pair of shoes, each pair of footbeds comprising a cushioning footbed and a supportive footbed.

Preferably, the container comprises a shoebox.

Preferably, the assembly further comprises a pair of forefoot inserts. Preferably, the assembly further comprises a pair of half length footbeds adapted for insertion within the footwear to cooperate with the footwear. Suitably, each half length footbed of the pair of half length footbeds may suitably be a cushioning half length footbed or a supportive half length footbed. The cushioning half length footbed is adapted for insertion within the footwear to cooperate with the footwear to thereby produce a soft, wide fitting. The supportive half length footbed is adapted for insertion within the footwear to cooperate with the footwear to thereby produce a supportive, wide fitting.

In yet another form, the invention resides in a method for providing a range of fittings and/or supports to a shoe of a particular size, the method comprising the steps of:
providing a plurality of footbeds, the footbeds comprising at least a cushioning footbed and a supportive footbed; and
inserting the cushioning footbed into the shoe to provide a soft feel; or
inserting the supportive footbed into the shoe to provide a support to reduce or prevent excessive pronation.

Preferably, the method further comprises the steps of:
providing a forefoot insert; and
inserting the forefoot insert into the shoe beneath the cushioning full length footbed or the supportive full length footbed to provide a narrower fitting.

Preferably, the method further comprises the steps of:
providing a half length insert, the half length insert comprising a supportive half length footbed or a cushioning half length footbed;
inserting the half length insert into the shoe to provide a wide fitting.

In another form, the invention provides an assembly for providing footwear with a plurality of removable inserts comprising:
an item of footwear;
a cushioning footbed adapted for insertion within the footwear to cooperate with the footwear to thereby produce a soft feel; and
a supportive footbed adapted for insertion within the footwear to cooperate with the footwear to thereby produce a supportive or stabilised fitting,
wherein in use the cushioning footbed is inserted into the item of footwear to provide a soft feeling underfoot; or
the supportive footbed is inserted into the item of footwear to provide a stabilised or supportive fit to reduce or prevent excessive pronation.

Preferably, the cushioning footbed comprises a cushioning full length footbed or a cushioning half length footbed.

Preferably, the supportive footbed comprises a supportive full length footbed or a supportive half length footbed.

Preferably, the assembly comprises at least one cushioning footbed and at least one supportive footbed. Preferably, the assembly comprises at least one of:
a cushioning full length footbed adapted for insertion within the footwear to cooperate with the footwear to thereby produce a soft feel, wherein in use the cushioning full length footbed is inserted into the item of footwear to provide a soft feeling underfoot; and
a cushioning half length footbed adapted for insertion within the footwear to cooperate with the footwear to thereby produce a soft feel for a wide foot, wherein in use the cushioning half length footbed is inserted into the item of footwear to provide a soft feeling underfoot for a wide foot,
and at least one of:
   a supportive full length footbed adapted for insertion within the footwear to cooperate with the footwear to thereby produce a supportive feel, wherein in use the supportive full length footbed is inserted into the item of footwear to provide a support to reduce or prevent excessive pronation; and
   a supportive half length footbed adapted for insertion within the footwear to cooperate with the footwear to thereby produce a supportive wide fitting, wherein in use the supportive half length footbed is inserted into the item of footwear to provide a supportive wide fit to reduce or prevent excessive pronation.

In another form, the invention resides in an assembly for providing footwear with a plurality of removable and interchangeable inserts comprising:
an item of footwear;
a supportive full length footbed insert adapted for insertion within the footwear to cooperate with the footwear to thereby produce a support to reduce or prevent excessive pronation; and
a forefoot insert adapted for insertion beneath the supportive full length footbed within the footwear so that the supportive full length footbed and the forefoot insert cooperate with the footwear to produce a narrow fitting in combination with the supportive feel of the supportive footbed.

Further features and advantages of the present invention will become apparent from the following detailed description.

### BRIEF DESCRIPTION OF THE DRAWINGS

By way of example only, preferred embodiments of the invention will be described more fully hereinafter with reference to the accompanying figures, wherein:

FIG. 1 illustrates a footbed assembly comprising a supportive full length footbed, a cushioning full length footbed and a forefoot insert;

FIG. 2 illustrates a top view of the forefoot insert;

FIG. 3 illustrates a top view of the cushioning full length footbed;

FIG. 4 illustrates a view of the side of the cushioning full length footbed with the forefoot insert placed underneath;

FIG. 5 illustrates the cushioning full length footbed inserted into a shoe;

FIG. 6 illustrates a top view of the supportive full length footbed;

FIG. 7 illustrates a view of the side of the supportive full length footbed;

FIG. 8 illustrates a view of the side of the supportive full length footbed with the forefoot insert placed underneath;

FIG. 9 illustrates the supportive full length footbed inserted into a shoe;

FIG. 10 illustrates a cross section of the shoe having the supportive full length footbed inserted therein; and

FIG. 11 illustrates a cross section of the shoe having the supportive full length footbed and the forefoot insert inserted therein;

FIG. 12 illustrates a top view of a supportive half length footbed;

FIG. 13 illustrates a perspective view of the supportive half length footbed;

FIG. 14 illustrates a side view of the supportive half length footbed;

FIG. 15 illustrates a perspective view of a second embodiment of a cushioning full length footbed;

FIG. 16 illustrates a side view of the cushioning full length footbed of FIG. 15;

FIG. 17 illustrates an elevated side view of the cushioning full length footbed of FIGS. 15 and 16;

FIG. 18 illustrates a lateral cross-section of a midsole of shoe in accordance with an embodiment of the present invention; and

FIG. 19 illustrates a container having a pair of shoes, a pair of cushioning full length footbeds and a pair of supportive full length footbeds.

Preferred features, embodiments and variations of the invention can be discerned from the following Detailed Description which provides sufficient information for those skilled in the art to perform the invention. The Detailed Description and associated figures are not to be regarded as limiting the scope of the preceding Summary of the Invention in any way.

### DETAILED DESCRIPTION OF PREFERRED EMBODIMENTS

The present invention generally relates to a footwear insert assembly having a cushioning footbed and a supportive footbed for insertion within a shoe to reduce or prevent excessive pronation of a person during weight bearing activities, including walking or running, for example.

More particularly, embodiments of the invention relate to a footbed assembly insertable into an item of footwear, the assembly comprising at least two full length footbeds which are interchangeably insertable into the item of footwear. A first footbed is substantially flat, offering little or no contouring or arch/heel support. A second footbed is a supportive footbed designed to reduce or prevent excessive pronation. Some embodiments of the invention provide a smaller forefoot insert positionable under either of the two full length footbeds to lift part of the full length footbed in the footwear to provide a tighter fit.

Embodiments of the present invention relate to a footwear insert system having a posted footbed for insertion within a shoe to reduce or prevent excessive pronation of a person during walking or running.

More particularly, embodiments relate to a footbed assembly insertable into footwear, the assembly comprising a full length footbed designed to reduce or prevent excessive pronation and a smaller forefoot insert positionable under the full length footbed to lift part of the full length footbed in the footwear to provide a tighter fit.

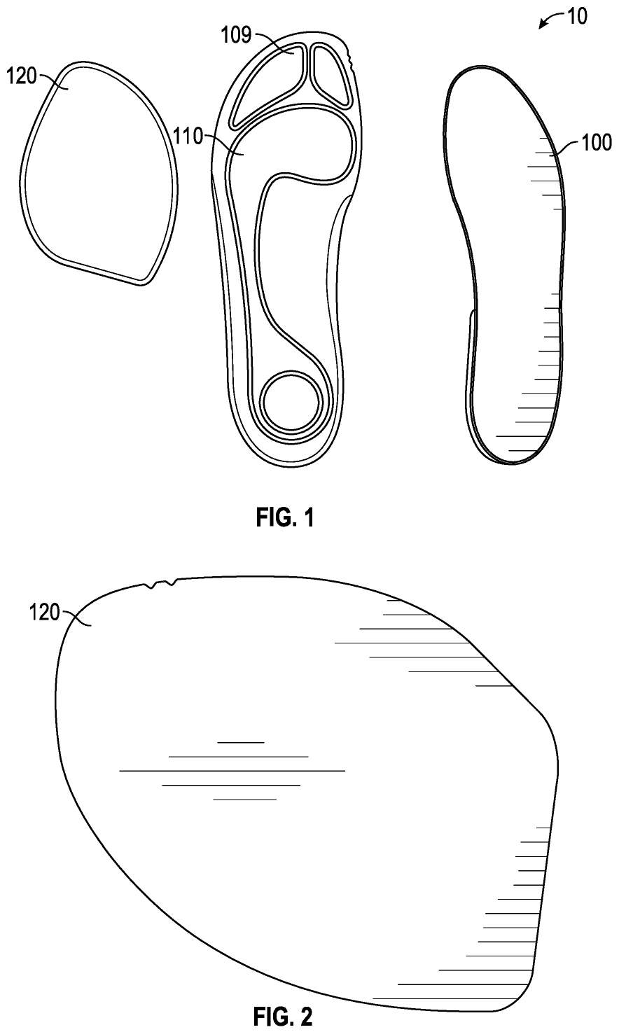

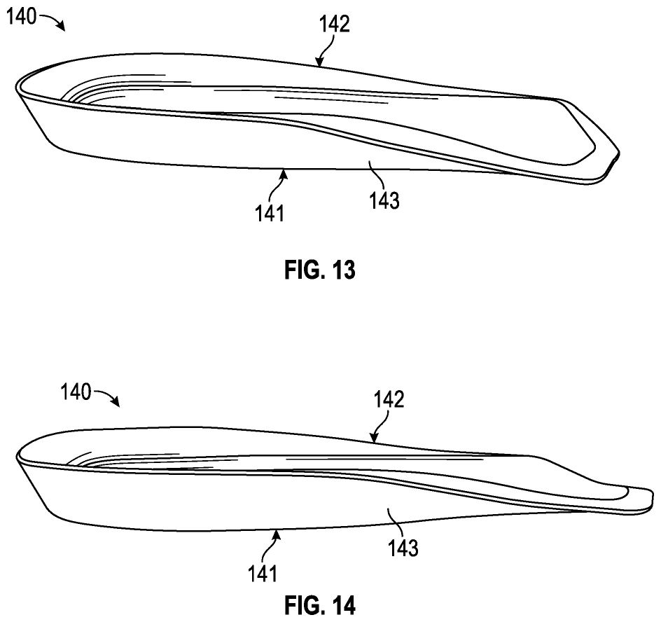

With reference to FIG. 1, there is shown a footbed assembly 10 comprised of three parts: a cushioning full length footbed 100, a supportive full length footbed 110 and a forefoot insert 120. While the cushioning full length footbed 100, the supportive full length footbed 110 (shown with underside facing out of page) and the forefoot insert 120 are all designed for right feet, it will be readily understand that the footbeds 100, 110 and insert 120 could be mirrored for use with left feet. Left feet examples of the cushioning full length footbed 100a and the supportive full length footbed 110a can be seen in FIG. 13 along with a left shoe 130a.

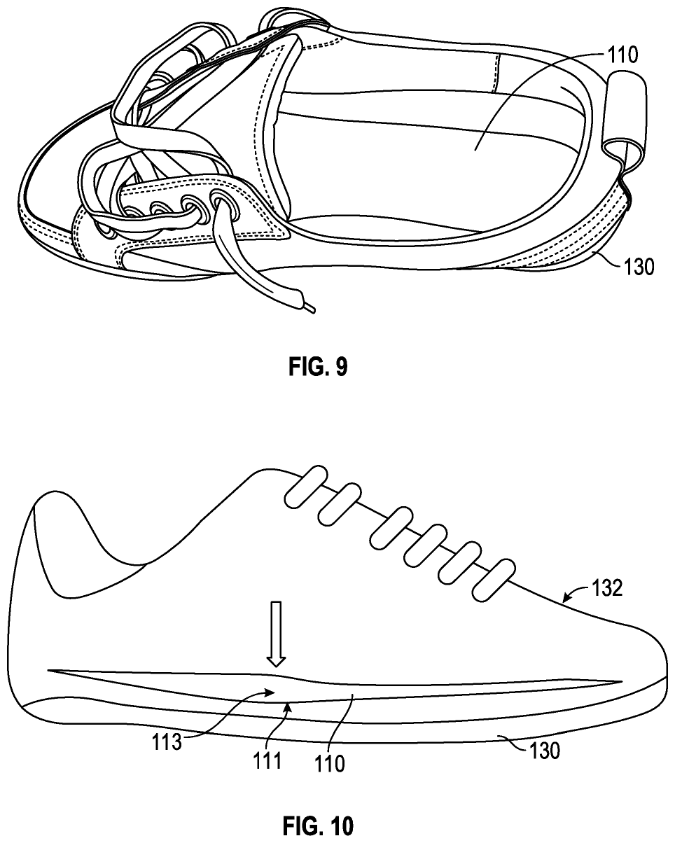

The assembly 10 is designed to be used with an item of footwear (such as right shoe 130 shown in FIGS. 5 and 9) such that the cushioning full length footbed 100 and the supportive full length footbed 110 (both optionally used with the forefoot insert 120 as required) are interchangeably insertable into the item of footwear to achieve a desired fit. In some embodiments, the shoe will be a neutral shoe (i.e. a shoe having no arch or heel support in the sole) or a shoe having a built-up or supportive sole (preferably a supportive midsole).

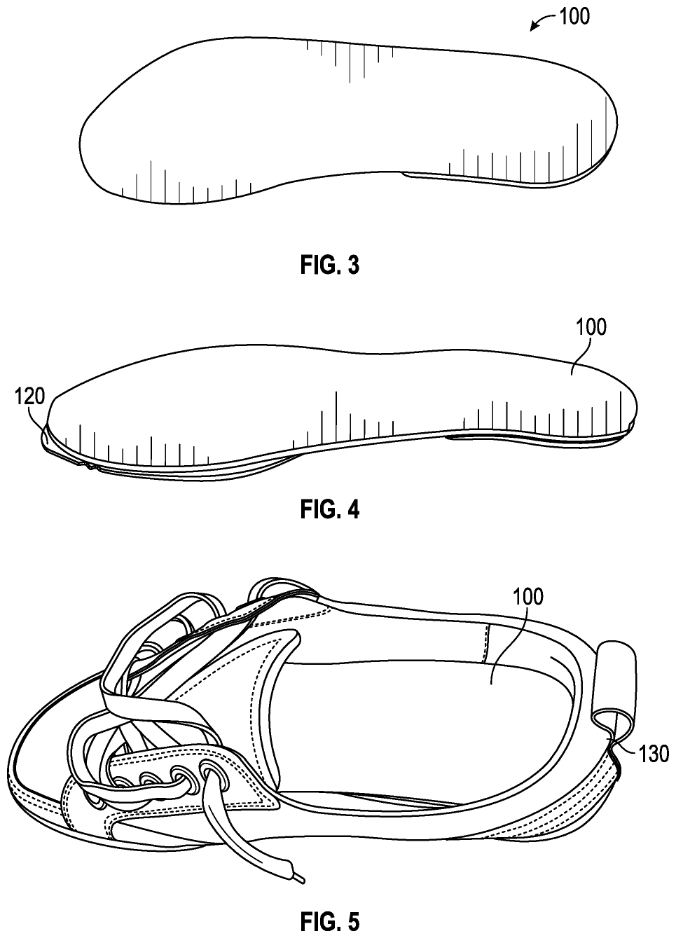

The cushioning full length footbed 100 is designed for a person with a neutral (or substantially neutral) foot who is not prone to excessive pronating and therefore does not require assistance or built-up cushioning in certain areas of the footbed. More particularly, the cushioning footbed 100 is a “comfort” or “soft, minimalist” footbed which feels soft and may be cushioned or have a cushioning effect for a user but does not provide any specific or tailored heel or arch support. The substantially flat nature and design of the cushioning footbed 100 (relative to the supportive footbed 110) can be more clearly seen in FIGS. 3 and 4. When inserted into a shoe (such as shoe 130 shown in FIG. 5, for example), the cushioning footbed 100 provides a standard width fitting.

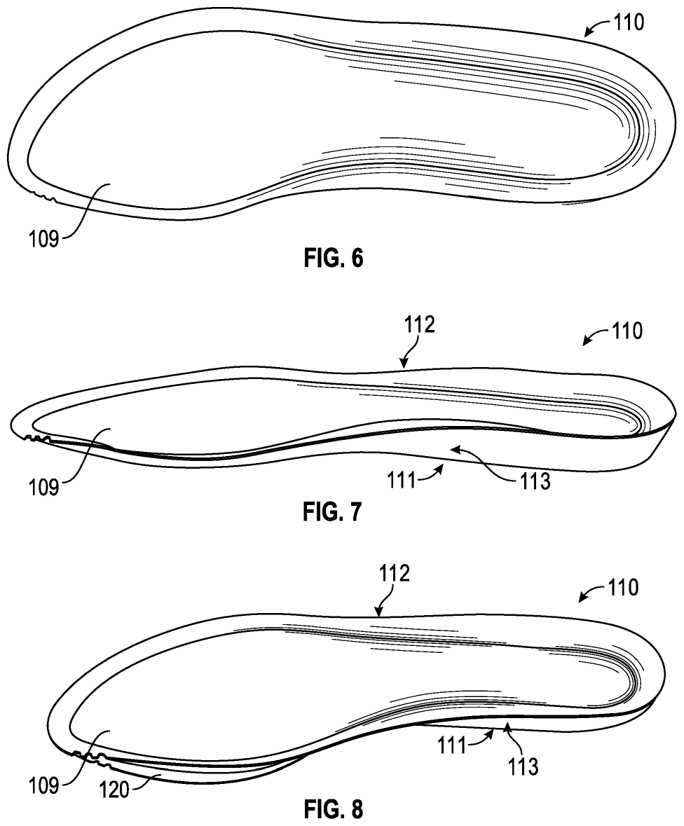

The supportive full length footbed 110 (also shown in FIG. 6-9) is a “control” or “supportive” footbed and includes a supported medial side 111 (opposite a lateral side 112) and is designed for a user who has a pronating foot tending toward overpronation or “flat footedness”. In use, the support 113 (comprising a built up or supporting portion) on the medial side 111 of the footbed 110 supports the arch and reduces or prevents the foot from excessively pronating to mitigate stress and/or injuries.

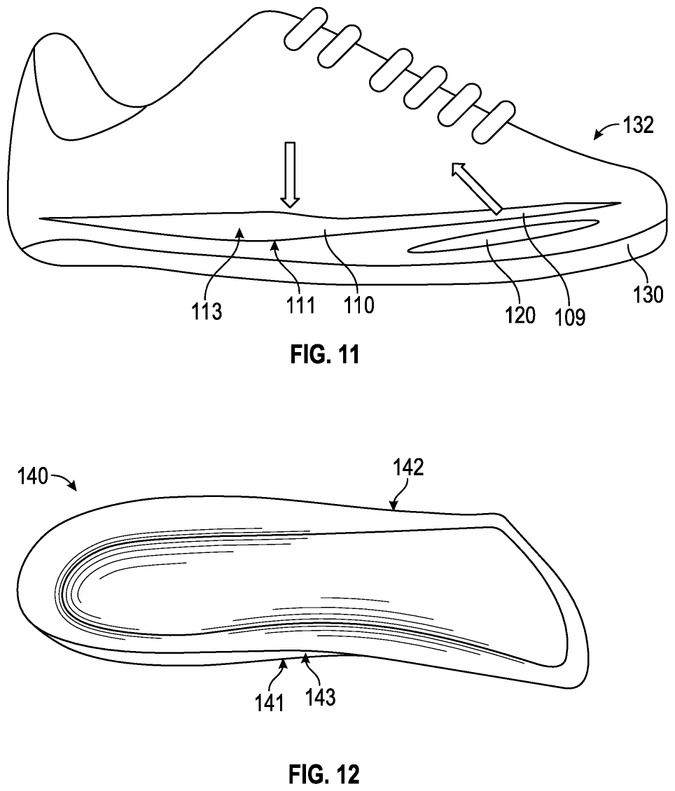

With particular reference to FIG. 7, FIG. 10 and FIG. 11, the built up support 113 on the medial side 111 reduces and/or prevents the flattening of the arch of the foot which occurs during overpronation.

In the embodiment illustrated in FIG. 10, as the shoe 130 worn by a person strikes the ground or a surface, the arch of the foot of the person wearing the shoe exerts downward force (as indicated by the downward arrow) on the support 113 on the medial side 111. As the support 113 is made of a sufficiently rigid and resilient material, such as foam or rubber, the support 113 does not fully flatten out, thereby supporting the arch and preventing the overpronation.

The embodiment in FIG. 11 is similar to the embodiment shown in FIG. 10 but additionally includes the additional forefoot insert 120 which is positioned under the supportive full length footbed 110 and causes the front part 109 of the footbed 110 to be deformed or lifted upwardly (as indicated by the upward arrow) to also provide the tighter fitting.

The forefoot insert 120, which can also be seen in FIG. 4 (in use with the cushioning footbed 100), FIG. 8 (in use with the supportive footbed 110) and FIG. 2, is designed to be used in conjunction with either of the cushioning full length footbed 100 or the supportive full length footbed 110 and provides a better, tighter fit for narrower feet. The forefoot insert 120 can be attached to a footbed 100, 110 by any suitable means, including using an adhesive as an example.

By inserting the forefoot insert 120 in a shoe beneath either of the footbeds 100, 110, it is possible to provide a tighter fit while maintaining the standard width footbed. The forefoot insert 120, when inserted beneath footbed 110, for example, causes the forward or front part 109 of the footbed 110 to be lifted upwardly (toward the top 132 of the shoe 130) to provide the tighter fit. Similarly, when inserted beneath footbed 100, the forefoot insert 120 has the same effect. As a result, a variety of different fittings can be provided in the same shoe using various combinations of the footbeds 100, 110 and forefoot insert 120.

The forefoot insert 120 also provides extra forefoot cushioning in addition to adjusting the fit of the shoe.

The width of a shoe is typically indicated as between A-E and is typically based on the width of the foot. Common step sizes are 3/16 of an inch. A particular shoe range can, as an example, be classified as a “C” width fitting but, using the provided forefoot insert 120, can be easily converted to a tighter fitting.

Turning to FIG. 12-14, there is shown a supportive half length footbed 140. Similar to the supportive full length footbed 110, the supportive half length footbed 140 includes a supported medial side 141 (opposite a lateral side 142) and is designed for a user who has a pronating foot tending toward overpronation or “flat footedness”. In use, the support 143 on the medial side 141 of the supportive half length footbed 140 supports the arch and reduces or prevents the foot from excessively pronating to mitigate injuries by supporting the arch of the foot and preventing it from flattening out which is typical during the overpronation motion. While not shown, a cushioning half length footbed can also be provided. Supportive half length footbed 140 is shown in a left foot configuration but could be easily and readily provided in a right foot configuration.

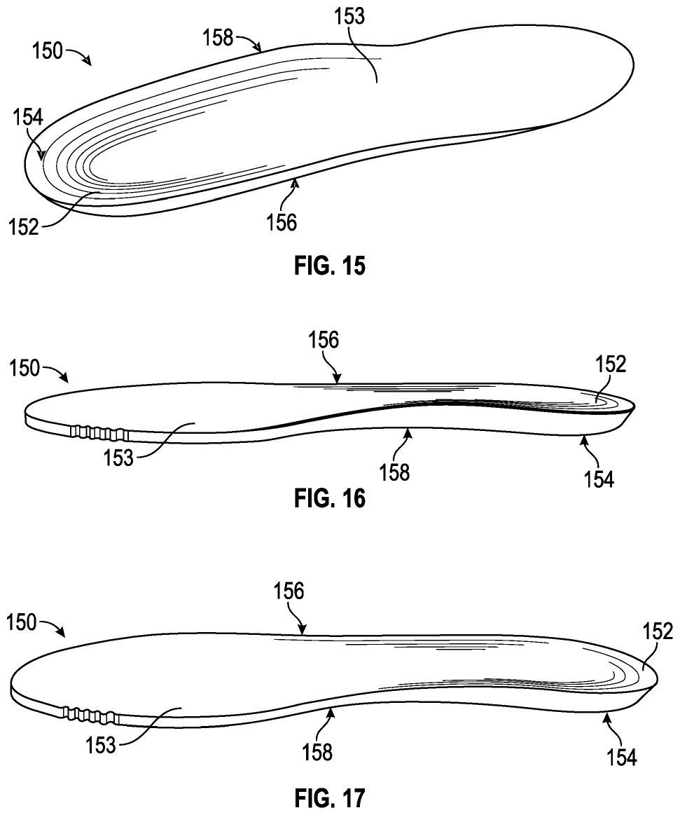

With reference to FIG. 15-17, there is illustrated a second embodiment of a cushioning footbed in the form of cushioning full length footbed 150. Cushioning full length footbed 150 is substantially similar to cushioning full length footbed 100 but includes a raised portion 152 connected to a flat portion 153 (which receives and supports the underside of the foot) and extending around the heel portion 154 and along a part of the lateral side 156 and the medial side 158. This raised portion 152 provides additional cushioning for the heel, arch and foot of a wearer but does not provide any support for the heel or arch to prevent or reduce pronation.

The cushioning full length footbed 150 is designed for a person with a neutral (or substantially neutral) foot who is not prone to excessive pronating and therefore does not require assistance or built-up cushioning in certain areas of the footbed. More particularly, the cushioning footbed 150 is a “comfort” or “soft, minimalist” footbed which feels soft and may be cushioned or have a cushioning effect for a user but does not provide any specific or tailored heel or arch support. The substantially flat nature and design of the cushioning footbed 150 (relative to the supportive footbed 110) can be more clearly seen in FIGS. 16 and 17. When inserted into a shoe (such as shoe 130 shown throughout the specification, for example), the cushioning footbed 150 provides a standard width fitting.

The cushioning full length footbed 100 and cushioning full length footbed 150 can be used interchangeably with any of the embodiments described herein.

While cushioning full length footbed 150 is shown only in a right foot configuration, it could be easily and readily adapted for a left foot configuration.

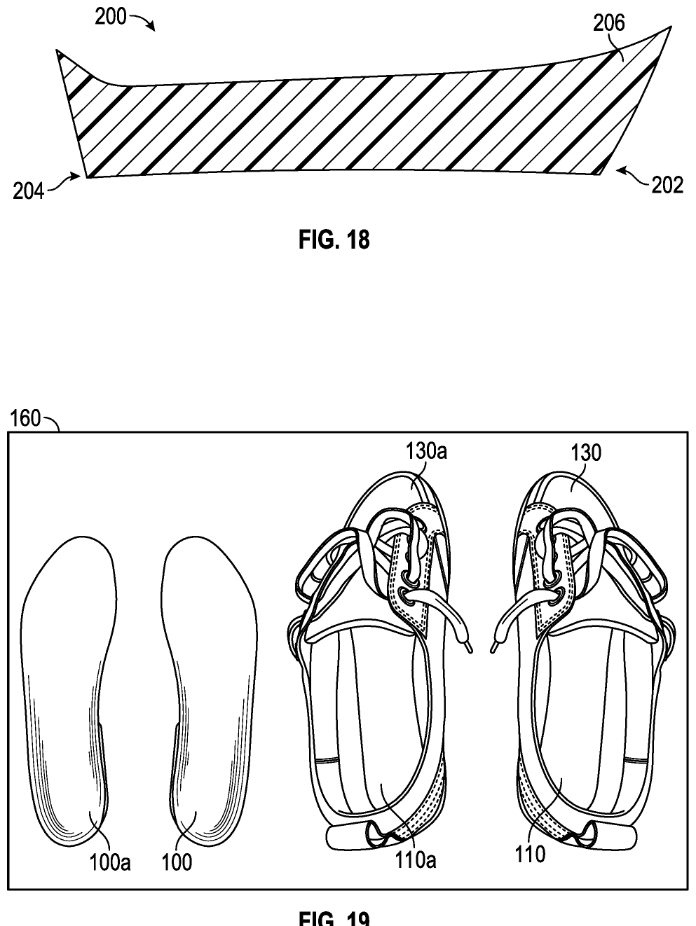

Another embodiment of the item of footwear in the form of a supportive midsole 200 (that is shown in a lateral cross-section) for a left shoe is shown in FIG. 18.

The supportive midsole 200 comprises a supported medial side 202 (opposite a lateral side 204) and is designed for a user who has a pronating foot tending toward overpronation or “flat footedness”. During wear, the support 206 (comprising a built up or supporting portion) on the medial side 202 of the midsole 200 supports the arch and reduces or prevents the foot from excessively pronating to mitigate stress and/or injuries. In use in accordance with embodiments of the invention, the midsole 200 forms part of the item of footwear or shoe (such as shoe 130, for example) that is used in conjunction with the cushioning full length footbed and supportive full length footbed. The supportive midsole 200 is particularly useful for wearers who have excessive pronation that can be mitigated by the supportive midsole 200, which provides base support, in combination with a cushioning full length footbed (such as cushioning full length footbeds 100, 150) in low impact activities (e.g. walking) or a supportive full length footbed (such as supportive full length footbed 120) for high impact activities (e.g. running or sport). It will be appreciated that the midsole 200 can be mirrored for a right shoe.

Referring now to FIG. 19, there is shown a container 160 (the container could be a shoebox or any other suitable container) having a pair of shoes (right shoe 130 and left shoe 130a), a pair of cushioning full length footbeds 100, 100a (left and right, respectively) and a pair of supportive full length footbeds 110, 110a, which are also left and right and inserted into shoes 130, 130a, respectively. In some embodiments, a pair of forefoot inserts (such as forefoot inserts 120) and/or a pair of half length footbeds (such as supportive half length footbeds 140) could also be provided in the container 160.

The invention allows a shoe to have a range of fitting choices for the customer without requiring the purchase of a different shoe or footbed for each desired fit.

It is envisioned that, in one embodiment, a pair of shoes in a shoebox will be purchased and the shoebox will contain a cushioning full length footbed to be worn for standard width feet which have a neutral foot, a supportive full length footbed to be worn for standard width feet which have a pronating foot. In use, the user would be able to use the cushioning full length footbed and the supportive full length footbed interchangeably (i.e. remove the cushioning full length footbed and replace it with the supportive full length footbed, and vice versa).

Furthermore, each shoe can be individually fitted. For example, a customer may pronate with their left foot but not their right foot. In this situation, the customer can insert the supportive full length footbed in the left shoe so that the left foot cannot excessively pronate, and insert the cushioning full length footbed in the right shoe for the right foot which does not require support or correction. Of course, any combination of the inserts/footbeds described throughout the specification can be provided with an individual shoe or pair of shoes. For example, the shoebox may also contain a forefoot insert that can be positioned underneath either of the footbeds in a forward part of the shoe to provide a tighter fit for narrower feet

The inserts/footbeds are particularly suitable for use with exercise shoes or running shoes but can be used with any type of shoe. In this regard, the shoe can remain comfortable while also preventing excessive pronation while walking or running when necessary.

Embodiments of the present invention offer a number of advantages. In one example, embodiments of the present invention enable users to adjust the level of foot support and feel of a single pair of shoes by simply changing the footbeds as required for use. More particularly, a person may wish to have a shoe that minimises pronation when they run, so they insert the supportive footbed, but that person also prefers a shoe with only cushioning for walking or during everyday use and wear, so they insert the cushioning footbed. Thus, embodiments of the invention provide them with the ability to customise the level of support provided by their shoe on an ongoing basis.

Aside from the ability to customise a shoe for specific uses, the user can also adjust a shoe based on their own personal preference for the “feel” of a shoe. In particular, a customer may like the look and design of a particular shoe and, using the present invention, can modify the feeling of the fit of the preferred shoe by interchanging the footbeds as desired to fit their personal preferences.

In addition, stockists or retailers will not need to stock two pairs of the same shoes one pair having the high control/supportive footbed and the other pair having the cushioning footbed. Embodiments of the present invention will allow retailers to simply have one shoe design that can be customised to the customer's desires. Advantageously, this minimises the amount of stock stores have to purchase and stock/display.

The term “footbed” used through the specification may comprise insoles, inserts and the like.

As used herein, the term “cushioning footbed” and the variations thereof (e.g. full length or half length) refer to a footbed that is substantially flat and soft, providing little or no arch support beyond that inherently provided by the footbed. In this regard, the cushioning footbed may comprise a raised portion about the heel and/or portions of the arch on both the medial side and the lateral side of the footbed. The cushioning footbed provides a normal fitting for a foot and a soft, minimalistic feeling for a user during wearing and use.

As used herein, the term “supportive footbed” and the variations thereof (e.g. full length or half length) refer to a footbed that provides arch support to reduce or eliminate excessive pronation. The supportive footbed provides a supportive feel for a foot, for example by means of a thickening or built up portion of the footbed in a region corresponding to a wearer's arch. The supportive footbed also provides a supportive feeling for a user during wearing and use. A skilled addressee will appreciate that the supportive footbed may also be known as a “posted footbed”.

In this specification, adjectives such as first and second, left and right, top and bottom, and the like may be used solely to distinguish one element or action from another element or action without necessarily requiring or implying any actual such relationship or order. Where the context permits, reference to an integer or a component or step (or the like) is not to be interpreted as being limited to only one of that integer, component, or step, but rather could be one or more of that integer, component, or step, etc.

The above detailed description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiments will be apparent or relatively easily developed by those of ordinary skill in the art. The invention is intended to embrace all alternatives, modifications, and variations of the present invention that have been discussed herein, and other embodiments that fall within the spirit and scope of the above described invention.

In this specification, the terms ‘comprises’, ‘comprising’, ‘includes’, ‘including’, or similar terms are intended to mean a non-exclusive inclusion, such that a method, system or apparatus that comprises a list of elements does not include those elements solely, but may well include other elements not listed.

Throughout the specification and claims (if present), unless the context requires otherwise, the term “substantially” or “about” will be understood to not be limited to the specific value or range qualified by the terms.

1. An assembly for providing footwear with a plurality of removable and interchangeable footbeds, the assembly comprising:
an item of footwear;
a cushioning full length footbed adapted for insertion within the footwear to cooperate with the footwear to thereby produce a soft feel;
a supportive full length footbed adapted for insertion within the footwear to cooperate with the footwear to thereby produce a supportive feel;
a forefoot insert arranged for insertion beneath the cushioning full length footbed or the supportive full length footbed within the footwear so that the cushioning full length footbed or the supportive full length footbed and the forefoot insert cooperate with the footwear to produce a narrow fitting,
wherein, in use, the cushioning full length footbed and the supportive full length footbed are interchangeably insertable into the item of footwear,
in use, the cushioning full length footbed is inserted into the item of footwear to provide a soft feeling underfoot; or
the supportive full length footbed is inserted into the item of footwear to provide a support to reduce or prevent excessive pronation, and
the cushioning full length footbed or the supportive full length footbed with the forefoot insert beneath a forward portion thereof is inserted into the item of footwear to provide a narrow fit in combination with the soft feel of the cushioning full length footbed or the supportive feel of the supportive full length footbed.

2. The assembly according to claim 1, wherein the forefoot insert is attachable to the cushioning full length footbed or the supportive full length footbed.

3. The assembly according to claim 2, wherein the forefoot insert is adhesively attachable to the cushioning full length footbed or the supportive full length footbed.

4. The assembly according to claim 1, the assembly further comprising a half-length footbed adapted for insertion within the footwear to cooperate with the for wider feet.

5. The assembly according to claim 4, wherein the half length footbed comprises:
a cushioning half length footbed adapted for insertion within the footwear to cooperate with the footwear to thereby produce a soft fitting for wider feet; or
a supportive half length footbed adapted for insertion within the footwear to cooperate with the footwear to thereby produce a supportive fitting for wider feet.

6. The assembly according to claim 4, the assembly comprising a pair of half length footbeds comprising a cushioning half length footbed and a supportive half length footbed.

7. The assembly according to claim 1, wherein the supportive full length footbed comprises a medial side and a lateral side.

8. The assembly according to claim 7, wherein the supportive full length footbed comprises a support located along the medial side, the support comprising a cushioned or contoured portion adapted to support an arch of a foot in the item of footwear to reduce or prevent excessive pronation.

9. The assembly according to claim 1, wherein the item of footwear is a shoe.

10. An assembly for providing footwear with a plurality of removable and interchangeable footbeds, the assembly comprising:
a container;
a pair of shoes located in the container;
at least two pairs of removable and interchangeable footbeds to provide a user with at least two fitting choices for the same pair of shoes, each pair of footbeds comprising:
a cushioning full length footbed adapted for insertion within the footwear to cooperate with the footwear to thereby produce a soft feel; and
a supportive full length footbed adapted for insertion within the footwear to cooperate with the footwear to thereby produce a supportive feel; and
a pair of forefoot inserts, each forefoot insert arranged for insertion beneath the cushioning full length footbed or the supportive full length footbed within the footwear so that the cushioning full length footbed or the supportive full length footbed and the forefoot insert cooperate with the footwear to produce a narrow fitting
wherein, in use, the cushioning full length footbed and the supportive full length footbed are interchangeably insertable into the item of footwear, and
wherein in use the cushioning full length footbed is inserted into the item of footwear to provide a soft feeling underfoot; or
the supportive full length footbed is inserted into the item of footwear to provide a support to reduce or prevent excessive pronation, and
the cushioning full length footbed or the supportive full length footbed with the forefoot insert beneath a forward portion thereof is inserted into the item of footwear to provide a narrow fit in combination with the soft feel of the cushioning full length footbed or the supportive feel of the supportive full length footbed.

11. The assembly according to claim 10, the assembly further comprising a pair of half length footbeds, each half length footbed adapted for insertion within the footwear to cooperate with the footwear for wider feet.

12. The assembly according to claim 11, wherein each half length footbed of the pair of half length footbeds comprises:
a cushioning half length footbed adapted for insertion within the footwear to cooperate with the footwear to thereby produce a soft fitting for wider feet; or
a supportive half length footbed adapted for insertion within the footwear to cooperate with the footwear to thereby produce a supportive fitting for wider feet.

13. A method for providing a range of fittings and/or supports to an item of footwear of a particular size, the method comprising the steps of:
providing a plurality of interchangeable and removable footbeds, the footbeds comprising:
a cushioning full length footbed adapted for insertion within the footwear to cooperate with the footwear to thereby produce a soft feel; and
a supportive full length footbed adapted for insertion within the footwear to cooperate with the footwear to thereby produce a supportive feel;
providing a forefoot insert adapted for insertion beneath the cushioning full length footbed or the supportive full length footbed within the footwear so that the cushioning full length footbed or the supportive full length footbed and the forefoot insert cooperate with the footwear to produce a narrow fitting;
inserting the cushioning full length footbed into the shoe to provide a soft feel; or
inserting the supportive full length footbed into the shoe to provide a support to reduce or prevent excessive pronation; and
inserting the forefoot insert into the shoe beneath the cushioning full length footbed or the supportive full length footbed to provide a narrower fitting.

14. The method according to claim 13, the method further comprising the steps of:
providing a half length footbed adapted for insertion within the footwear to cooperate with the footwear for wider feet, the half length insert comprising:
a supportive half length footbed adapted for insertion within the footwear to cooperate with the footwear to thereby produce a supportive fitting for wider feet; or
a cushioning half length footbed adapted for insertion within the footwear to cooperate with the footwear to thereby produce a soft fitting for wider feet; and
inserting the half length footbed into the shoe to provide fitting for wider feet.
